(19) United States
(12) Patent Application Publication
(10) Pub. No.: US 2011/0312023 A1
(43) Pub. Date: Dec. 22, 2011

(54) Integrated Technology of Algae Bio-Diesel and Algae Ferment Reuse

(76) Inventors: Jiunn-Jye Chuu, Xindian City (TW); Shun-Lai Li, Tainan City (TW); Hsiao-Hui Hsieh, Yongkang City (TW); Chia-Hui Cheng, Rende Township (TW)

(21) Appl. No.: 12/818,278

(22) Filed: Jun. 18, 2010

(51) Int. Cl. C12P 1/00

(52) U.S. Cl. 435/41

(57) ABSTRACT

An integrated technology of algae bio-diesel and algae ferment reuse includes providing algae, placing the algae into an optical reactor to grow the algae into rich algae, abstracting the rich algae to form abstracted algae and fat, placing the abstracted algae into a ferment tank so that the abstracted algae are decomposed and fermented to form a fermented substance and a fermented liquid, and making the fermented substance and the fermented liquid into a by-product. Thus, the algae produce carbon dioxide during the fermenting process, so that the carbon dioxide produced by the abstracted algae in the ferment tank is supplied to the algae in the optical reactor to form a breathing cycle between the optical reactor and the ferment tank so as to promote the growth of the algae simultaneously.

### BACKGROUND OF THE INVENTION

1. Field of the Invention

The present invention relates to an integrated technology and, more particularly, to an integrated technology of algae bio-diesel and algae ferment reuse.

2. Description of the Related Art

A bio-diesel fuel is usually made of crops, such as corns, soybeans and the like. However, the bio-diesel fuel made of crops easily causes lack of foodstuff in the world. Another bio-diesel fuel is made of fat contained in the algae. In growth of the algae, the algae is placed into an optical reactor to proceed a photo-synthesis process so that the algae produces a high content of fat during the photo-synthesis process. At this time, the algae contained in the optical reactor absorbs carbon dioxide and produces oxygen during the photo-synthesis process. However, the manufacturer has to supply the carbon dioxide to the algae from an external supply device to promote growth of the algae, thereby causing inconvenience in growth of the algae, and thereby greatly increasing the costs of production.

### BRIEF SUMMARY OF THE INVENTION

In accordance with the present invention, there is provided a method for growing and fermenting algae, comprising a first step of providing algae, a second step of placing the algae into an optical reactor to proceed a photo-synthesis process so as to grow the algae into rich algae with a high content of fat during the photo-synthesis process, a third step of abstracting the rich algae to form abstracted algae and fat, a fourth step of placing the abstracted algae into a ferment tank to ferment the abstracted algae so that the abstracted algae are decomposed and fermented to form a fermented substance and a fermented liquid, and a fifth step of making the fermented substance and the fermented liquid into a by-product.

The abstracted algae in the ferment tank produce carbon dioxide during the fermenting process. The carbon dioxide produced by the abstracted algae in the ferment tank during the fermenting process is supplied to the algae in the optical reactor to form a breathing cycle between the optical reactor and the ferment tank so as to promote growth of the algae simultaneously.

Preferably, the rich algae are placed into and abstracted by an abstracting device.

Preferably, the by-product includes a healthy foodstuff, a fertilizer and an organic liquid feedstuff.

Preferably, the healthy foodstuff and the fertilizer are made of the fermented substance.

Preferably, the organic liquid feedstuff is made of the fermented liquid.

According to the primary objective of the present invention, the algae produce carbon dioxide during the fermenting process, so that the carbon dioxide produced by the abstracted algae in the ferment tank during the fermenting process is supplied to the algae in the optical reactor to form a breathing cycle between the optical reactor and the ferment tank so as to promote the growth of the algae simultaneously.

According to another objective of the present invention, the abstracted algae in the ferment tank supply the carbon dioxide during the fermenting process so that the carbon dioxide are supplied to the algae successively without needing an external supply of carbon dioxide so as to facilitate the growth of the algae and to decrease the costs of production largely.

Further benefits and advantages of the present invention will become apparent after a careful reading of the detailed description with appropriate reference to the accompanying drawings.

### BRIEF DESCRIPTION OF THE SEVERAL VIEWS OF THE DRAWING(S)

FIG. 1 is a flow chart of an integrated technology of algae bio-diesel and algae ferment reuse in accordance with the preferred embodiment of the present invention.

### DETAILED DESCRIPTION OF THE INVENTION

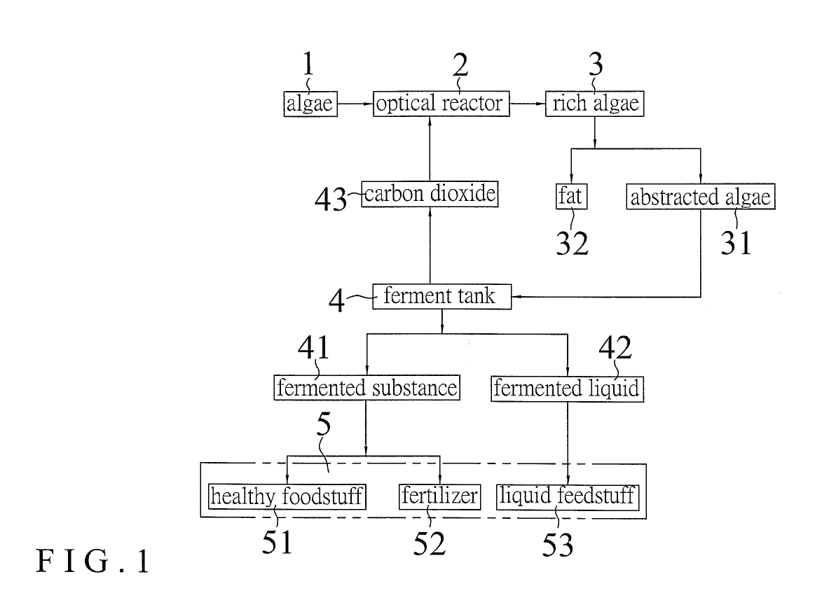

Referring to FIG. 1, an integrated technology of algae bio-diesel and algae ferment reuse in accordance with the preferred embodiment of the present invention comprises a first step of providing algae 1, a second step of placing the algae 1 into an optical reactor 2 to proceed a photo-synthesis process so as to grow the algae 1 into rich algae 3 with a high content of fat during the photo-synthesis process, a third step of abstracting the rich algae 3 to form abstracted algae 31 and fat 32, a fourth step of placing the abstracted algae 31 into a ferment tank 4 to ferment the abstracted algae 31 so that the abstracted algae 31 are decomposed and fermented to form a fermented substance 41 and a fermented liquid 42, and a fifth step of making the fermented substance 41 and the fermented liquid 42 into a by-product 5.

In the first step, the algae are single-cell algae that can produce a large amount of fat to make a bio-diesel fuel. In the second step, the optical reactor 2 is used to cultivate the algae 1 by controlling the growth environment conditions of the algae 1, including the sunlight, the carbon supply, the temperature, the PH value and the like, so as to increase the fat content of the algae 1. In the third step, the rich algae 3 are placed into and abstracted by an abstracting device. In addition, in the third step, the abstracted algae 31 contain rich polysaccharide and protein, and the fat 32 can be converted into a bio-diesel fuel. In the fourth step, the abstracted algae 31 in the ferment tank 4 produce carbon dioxide 43 during the fermenting process. In such manner, the carbon dioxide 43 produced by the abstracted algae 31 in the ferment tank 4 is supplied to the algae 1 in the optical reactor 2 during the photo-synthesis process. In the fifth step, the by-product 5 includes a healthy foodstuff 51, a fertilizer 52 and an organic liquid feedstuff 53, wherein the healthy foodstuff 51 and the fertilizer 52 are made of the fermented substance 41, and the organic liquid feedstuff 53 is made of the fermented liquid 42.

In practice of the integrated technology of algae bio-diesel and algae ferment reuse in accordance with the preferred embodiment of the present invention, the algae 1 are initially placed into the optical reactor 2 to proceed the photo-synthesis process so that the algae 1 are grown to form rich algae 3 with a high content of fat during the photo-synthesis process. At this time, the algae 1 in the optical reactor 2 absorb carbon dioxide the photo-synthesis process. Then, the rich algae 3 are placed in and abstracted by the abstracting device to form abstracted algae 31 and fat 32. At this time, the abstracted algae 31 contain rich polysaccharide and protein, and the fat 32 can be converted into a bio-diesel fuel. Then, the abstracted algae 31 are placed into and fermented in a ferment tank 4 so that the abstracted algae 31 are decomposed and fermented to form a fermented substance 41 and a fermented liquid 42. At this time, the abstracted algae 31 in the ferment tank 4 produce carbon dioxide 43 during the fermenting process. Finally, the fermented substance 41 and the fermented liquid 42 are made into a by-product 5. In such a manner, the carbon dioxide 43 produced by the abstracted algae 31 in the ferment tank during the fermenting process is supplied to the algae 1 in the optical reactor 2.

Accordingly, the algae produce carbon dioxide during the fermenting process, so that the carbon dioxide 43 produced by the abstracted algae 31 in the ferment tank 4 during the fermenting process is supplied to the algae 1 in the optical reactor 2 to form a breathing cycle between the optical reactor 2 and the ferment tank 4 so as to promote the growth of the algae simultaneously. In addition, the abstracted algae 31 in the ferment tank 4 supply the carbon dioxide 43 during the fermenting process so that the carbon dioxide 43 are supplied to the algae successively without needing an external supply of carbon dioxide so as to facilitate the growth of the algae and to decrease the costs of production largely.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

1. A method for growing and fermenting algae, comprising:
a first step of providing algae;
a second step of placing the algae into an optical reactor to proceed a photo-synthesis process so as to grow the algae into rich algae with a high content of fat during the photo-synthesis process;
a third step of abstracting the rich algae to form abstracted algae and fat;
a fourth step of placing the abstracted algae into a ferment tank to ferment the abstracted algae so that the abstracted algae are decomposed and fermented to form a fermented substance and a fermented liquid; and
a fifth step of making the fermented substance and the fermented liquid into a by-product.

2. The method of claim 1, wherein
the abstracted algae in the ferment tank produce carbon dioxide during the fermenting process;
the carbon dioxide produced by the abstracted algae in the ferment tank during the fermenting process is supplied to the algae in the optical reactor to form a breathing cycle between the optical reactor and the ferment tank so as to promote growth of the algae simultaneously.

3. The method of claim 1, wherein in the third step, the rich algae are placed into and abstracted by an abstracting device.

4. The method of claim 1, wherein in the fifth step, the by-product includes a healthy foodstuff, a fertilizer and an organic liquid feedstuff.

5. The method of claim 4, wherein the healthy foodstuff and the fertilizer are made of the fermented substance.

6. The method of claim 4, wherein the organic liquid feedstuff is made of the fermented liquid.
